(19) United States
(12) Patent Application Publication
(10) Pub. No.: US 2024/0390402 A1
(43) Pub. Date: Nov. 28, 2024

(54) COMPOSITION

(71) Applicant: HYBRISAN LTD., Port Talbot, West Glamorga (GB)

(72) Inventors: Christopher MORTIMER, Port Talbot, West Glamorgan (GB); Lee BRIDGEMAN, Port Talbot, West Glamorgan (GB)

(21) Appl. No.: 18/700,691

(22) PCT Filed: Oct. 12, 2022
(86) PCT No.: PCT/GB2022/052578

§ 371 Date: Apr. 11, 2024

(30) Foreign Application Priority Data
Oct. 13, 2021 (GB) 2114628.7

(51) Int. Cl. A61K 31/695; A61K 8/41; A61K 8/898; A61K 31/14; A61P 17/02; A61Q 17/00

(52) U.S. Cl. CPC A61K 31/695; A61K 8/416; A61K 8/898; A61K 31/14; A61P 17/02; A61Q 17/005

(57) ABSTRACT

The present invention provides an antimicrobial composition comprising: (i) one or more water soluble organosilanes at a concentration of about 0.01% to about 0.4% w/v; (ii) one or more quarternary ammonium compounds at a concentration of about 0.01% to 0.5% w/v; and (iii) one or more non-ionic or amphoteric or sarcosine anionic surfactants at a concentration of about 0.05% to about 1% w/v. Methods of making the composition and methods of treating and/or preventing a skin and/or wound infection using the composition are also provided.

### FIELD OF INVENTION

The present invention relates to antimicrobial compositions, uses of the same and to methods for treating or preventing skin and/or wound infections.

### BACKGROUND TO THE INVENTION

The worldwide problem of bacterial resistance has grown to such an extent that several organisms (for example, Klebsiella pneumoniae, Escherichia coli and Staphylococcus aureus) have developed resistance to multiple different antibiotics. According to the Centres for Disease Control and Prevention (CDC), in 2013 there were over 2 million infections with antibiotic resistant microorganisms and over 23 thousand subsequent deaths from those infections.

Due to the continuing rise of antimicrobial resistance to antibiotics there is a need to conserve the use of antibiotics to situations where there are no effective alternative options, such as spreading local and systemic infections. Maintenance debridement and use of topical antimicrobials (antiseptics) have been shown to be more effective than antibiotics when treating open wounds. Antiseptics are effective through many mechanisms of action, unlike antibiotics, which makes the development of resistance to them unlikely.

However, despite the use of antiseptics, infection is the likeliest single cause of delayed healing in healing of open wounds, especially chronic open wounds. If neglected it can progress from contamination to colonization and local infection through to systemic infection, sepsis and multiple organ dysfunction syndrome, and it can be life-threatening. Infection in chronic wounds can be additionally complicated by the presence of biofilms.

A biofilm can be formed from any syntrophic consortium of microorganisms in which cells stick to each other and often also to a surface. These adherent cells become embedded within a slimy extracellular matrix that is composed of extracellular polymeric substances (EPSs). The cells within the biofilm produce the EPS components, which are typically a polymeric conglomeration of extracellular polysaccharides, proteins, lipids and DNA.

There is, as yet, no diagnostic for biofilm presence but it contributes to excessive inflammation and activation of immune complexes and complement, leading to a delay in healing. Control of biofilm is therefore a key part of chronic wound management but at present there are relatively few antiseptics that can effectively treat biofilms in wounds. This is further complicated by the need for the antiseptic agent to have little to no cytotoxicity so that it does not cause further harm to the patient's own cells and tissues. This can be quantified using the therapeutic index, which is a ratio between the safety and effectiveness of a drug. It is critical to get this balance right when selecting treatments for bacteria and biofilm as often efficacy comes with high levels of cytotoxicity.

Biofilms have been shown to exhibit increased tolerance to many antimicrobials and antibiotics (Salisbury et al 2018). Therefore, it cannot be assumed that antimicrobials effective against planktonic bacterial are effective against biofilm. This has resulted in a need for antimicrobials specifically formulated to combat biofilm.

There is therefore a need for novel antimicrobial compositions that are effective against a wide variety of microorganisms and especially bacteria, including antibiotic resistant bacteria and biofilms, and which also have low cytotoxicity.

### SUMMARY OF THE INVENTION

The present invention provides an antimicrobial composition comprising (i) one or more water soluble organosilanes at a concentration of about 0.01% to about 0.4% w/v or about 0.5% w/v; (ii) one or more quarternary ammonium compounds at a concentration of about 0.01% to 0.5% w/v; and (iii) one or more non-ionic or amphoteric or sarcosine anionic surfactants at a concentration of about 0.05% to about 1% w/v. Compositions of the invention have been demonstrated to have highly effective antimicrobial activity and to be capable of inhibiting and disrupting established biofilms while also demonstrating a low cytotoxicity.

The antimicrobial compositions can be incorporated into a wound irrigation solution, which can be used to cleanse all types of wounds. Chronic skin wounds are often coated with slough, necrotic tissue and/or microbial biofilms. These coatings are difficult to remove and lead to delays in wound healing. Compositions of the present invention can remove these barriers of wound healing through their antimicrobial and cleansing activity. Chronic wounds are often infected with microorganisms and contaminants that can delay healing, making this complex process longer. The components of the composition of the present invention provide a tailored formulation that includes antimicrobial substances, quaternary ammonium compounds, and a surfactant, which work in combination to aid in the removal and prevention of biofilm formation. The compositions have a broad range of antimicrobial properties and have been tested against Gram negative and positive wound microorganisms as wells multispecies biofilms, and are also effective against fungi such as Candida albicans.

### DESCRIPTION

The present invention provides an antimicrobial composition comprising: (i) one or more water soluble organosilanes at a concentration of about 0.01% to about 0.4% w/v or about 0.5% w/v; (ii) one or more quarternary ammonium compounds at a concentration of about 0.01% to 0.5% w/v; and (iii) one or more non-ionic or amphoteric or sarcosine anionic surfactants at a concentration of about 0.05% to about 1% w/v.

Without being bound by theory, it is believed the modes of antimicrobial action of each component work in combination through a number of mechanisms. Organosilanes and quarternary ammonium products are known to bind to the cell membrane of which results in the leakage of intracellular constituents. Organosilanes in particular are known only to work by a physical kill mechanism, which does not promote the development of drug resistant microorganisms. Further to the physical mechanism of quarternary ammonium compounds they are also known to inactivate energy-producing enzymes, denature essential cell proteins, induce autolytic enzyme activity and breakdown RNA material. Polymeric biguanides are also known to disrupt microbial cell membranes however they can also disrupt cellular metabolism, interfere with cellular function and bind to DNA causing chromosome condensation. It is believed that in combination these mechanisms are complimentary, with the quarternary ammonium compounds and polymeric biguanide bactericidal concentrations decreasing as a result of interference with the cell membrane which increases entry into the microbial cell, with the other discussed mechanisms leading to microbial death.

Organosilanes are known to be hygroscopic (i.e., absorb moisture) and on contact with water quickly react, which reduces efficacy of the organosilane and makes the composition go from clear to cloudy. This significantly reduces the biofilm efficacy of the composition. The present inventors have determined that non-ionic and/or cationic surfactants can stabilise the organosilane, with compositions remaining clear for extended periods. However, upon efficacy testing the inventors found that the addition of these surfactants could have a significant negative impact, completely inhibiting antimicrobial efficacy in some cases. The inventors therefore determined minimum concentrations of a range of surfactants that maintained stability of the composition whilst also maintaining biofilm efficacy. Based on this work the inventors have been able to identify concentration ranges for a number of effective and stable compositions.

The presence of surfactant(s) and a chelating agent increase the efficacy against biofilm due to a reduction in surface tension and through the binding of metal ions holding the EPS together. In combination, these can disrupt the EPS and allow the antimicrobial components to penetrate the biofilm, killing microbial cells. This activity avoids the need for high concentrations of antimicrobial components and therefore also helps to avoid problems with cytotoxicity. As noted above, the surfactant(s) also help to stabilise the composition without affecting the efficacy of the antimicrobial agent.

On combination with other constituents, hydrolyzed organosilanes are known to go through a condensation reaction forming products which are insoluble in water—this reduces their antimicrobial activity and results in the solution going from clear to hazy. This can be overcome through careful selection of surfactants, including quarternary ammonium compounds, to stabilise the organosilane at a broad pH range. Furthermore, the inclusion of a chelating agent, particularly EDTA, is pH dependent and also known to interact with surfactants which can result in precipitation. Careful selection of the EDTA salt and combination of ingredients has allowed this to be overcome.

Antimicrobial compositions of the present invention may kill and/or inhibit the growth of microorganisms including bacteria, fungi, algae, protozoa, viruses and sub-viral agents. The compositions may be microbicidal or microbiostatic and can be disinfectants or antiseptics. Antimicrobial compositions of the invention may be antibacterial, antifungal or antiparasitic. Antimicrobial compositions of the invention are preferably antibiofilm.

Water soluble organosilanes contain silicon-bonded hydrolysable groups, such as alkoxysilanes, which allow the organosilanes to covalently bond to substrates containing hydroxyl or other silicon-reactive groups. Organosilanes are therefore often used as coupling agents to improve the bonding of fillers to resins, e.g., in making fibreglass. Organosilanes can also be used as antimicrobial additives for surfaces and textiles and are widely formulated into antimicrobial coatings.

Lower concentrations of organosilane are desirable as this reduces the concentration of non-ionic or amphoteric surfactant required and also leads to a reduction in cytotoxicity. Non-ionic and amphoteric surfactants are known to “shield” the antimicrobial effect of quaternary ammonium compounds so a minimal concentration is required to ensure a high efficacy at low concentrations.

Water soluble organosilanes may have a general formula of:

A₃₋ₓBₓSiD [Formula 1]

wherein:
A is —OH or a hydrolyzable group, such as a halide like —Cl, —Br, and —I, alkoxy or alkoxyether, such as those of the formula —OR¹ and —OR^{2A}OR¹ where each R¹ is R² or hydrogen, R² is an alkyl group of from 1 to 4 carbon atoms such as methyl, ethyl, propyl, butyl or —CH₂CH₂CH₂(CH₃), with methyl being preferred, and R^{2A} is a divalent saturated hydrocarbon group of from 1 to 4 carbon atoms such as methylene, ethylene, propylene, butylene or —CH₂CH₂CH(CH₃)— with ethylene and propylene being preferred; amino such as —N(R¹)₂ such as —NHCH₃, —N(CH₃)₂, and —NCCH₂CH₂)₂, also including organosilazanes where two organosilanes are combined by a —NH-unit; acetoxy which is —OOCCH₃; acetamido which is —HNOCCH₃; and hydride which is —H, among others known in the art;
B is an alkyl group of from 1 to 4 carbon atoms, with methyl being preferred;
x has a value of 0, 1 or 2; and
D is a hydrocarbon group of from 1 to 4 carbon atoms, phenyl, or a nonionic or cationic, substituted-hydrocarbon group containing at least one oxygen or nitrogen group or salts of such substituted hydrocarbon groups.

Preferably, the water soluble organosilane is a quarternary ammonium silane. Quarternary ammonium silanes are known to have broad spectrum antimicrobial activities with low cytotoxicity. Quarternary ammonium silanes have functional end-OH groups on their surfaces which can be surface modified (by use of acids) to activate the —OH groups. The antimicrobial activity of quaternary ammonium silanes is derived from a —C₁₈H₃₇ lipophilic alkyl chain which penetrates and/or binds to bacterial cell walls and membranes causing autolysis, a mechanism known as “contact killing”. These compounds have shown their effectiveness in reduction of bacterial growth in a wide range of applications including textiles, medical devices, and dental materials.

The quaternary ammonium silane may be one or more of dimethyloctadecyl[3-(trimethoxysilyl)propyl]ammonium chloride and 3-(trihydroxysilyl)propyldimethyloctadecylammoniumchloride. Preferably, the quarternary ammonium silane is dimethyloctadecyl[3-(trimethoxysilyl)propyl]ammonium chloride.

The antimicrobial composition comprises one or more water soluble organosilanes at a concentration of about 0.01% to about 0.4% w/v or about 0.5% w/v, preferably at a concentration of about 0.05% to about 0.3% w/v or about 0.4% w/v. The composition may comprise one or more water soluble organosilanes at a concentration of about 0.1% w/v.

The antimicrobial composition additionally comprises one or more quaternary ammonium compounds. Said compounds are preferably water soluble and/or organic. Suitable quaternary ammonium compounds may be free of silicon atoms and contain at least one nitrogen-bonded hydrocarbon group of at least 8 carbon atoms. Suitable quaternary ammonium compounds may be selected from one or more of benzalkonium chloride (BAC), didecyldimethylammonium chloride (DDAC), benzethonium chloride, tetradonium bromide, cetrimonium bromide, laurtrimonium bromide, cetalkonium chloride and cetrimonium chloride.

The antimicrobial composition comprises one or more quaternary ammonium compounds at a concentration of about 0.01% to 0.5% w/v, preferably at a concentration of about 0.05% to about 0.4% w/v. The composition may comprise one or more quaternary ammonium compounds at a concentration of about 0.2% w/v.

The antimicrobial composition additionally comprises one or more non-ionic or amphoteric or sarcosine anionic surfactants.

Examples of nonionic surfactants include C₈ to C₁₈ alcohol ethoxylates; C₈ to C₁₈ fatty acid esters of sorbitan or polyethoxylated sorbitan such as the laurate, oleate, stearate, and palmitate esters of sorbitan and sorbitan anhydride; C₈ to C₁₈ fatty acid esters and amides such as PEG-5 cocoate, PEG-15 cocoate, PEG-4 dilaurate, PEG-32 dilaurate, PEG-3 cocamide, PEG-6 cocamide, PEG-11 cocamide, PEG 20 dioleate, PEG-6 isopalmitate, PEG-12 isostearate, PEG-3 lauramide, PEG-8 laurate, PEG-32 laurate, PEG-4 octanoate, PEG-7 oleamide, PEG-2 oleate, PEG-14 oleate, PEG-20 palmitate, PEG-14 stearate, and PEG-5 tallowamide; C₈ to C₁₈ fatty alcohols such as caprylic alcohol, lauryl alcohol, cetyl alcohol and stearyl alcohol; C₈ to C₁₈ diols such as tetramethyl decynediol and dimethyl octynediol, block copolymers of polyethylene oxide and polypropylene oxide; C₈ to C₁₈ fatty acid esters of glycerine such as glyceryl caprate, glyceryl isostearate, glyceryl laurate, glyceryl myristate and glyceryl oleate; ethoxylated and propoxylated C₈ to C₁₈ fatty alcohols such as ethoxylated and propoxylated lauryl alcohol; C₈ to C₁₈ fatty amine and amidoamine oxides such asodecylamine oxide, cocamine oxide, cocamidopropylamine oxide, myristamine oxide, myristamidopropylamine oxide, palmita mine oxide, and stearamine oxide; C₈ to C₁₈ fatty amides and alkanolamides such as cocamide, cocamide DEA, cocamide MEA, stearamide, stearamide DEA, stearamide MEA and stearamide MIPA; and C₈ to C₁₈ fatty alcohol ethoxylates, tetramethyl decynediol, and ethoxylated and pro-poxylated lauryl alcohol.

Examples of amphoteric surfactants include C₈ to C₁₈ sultaines such as coco-sultaine and cocamidopropyl hydroxysultaine; C₈ to C₁₈ fatty derivatives of amino acids such as cocamphocarboxyglycinate and lauram-phoglycinate, as well as the more preferred C₈ to C₁₈ alkyl betaines such as decyl betaine, coco-betaine, lauryl betaine, myristyl betaine and stearyl betaine; and C₈ to C₁₈ amidoalkyl betaines such as cocoamidoethyl betaine, cocamidopropyl betaine, lauramidopropyl betaine, myristamidopropyl betaine and oleamidopropyl betaine, stearamidopropyl betaine.

Examples of sarcosine anionic surfactants include C₈ to C₁₈ alkyl sarcosines and their alkali metal or ammonium salts such as sodium, potassium, lithium or ammonium C₈ to C₁₈ alkyl sarcosinates which include cocoyl sarcosine, lauroyl sarcosine, sodium lauroyl sarcosinate, potassium lauroyl sarcosinate, lithium lauroyl sarcosinate, ammonium lauroyl sarcosinate, sodium cocoyl sarcosinate and potassium cocoyl sarcosinate. If a C₈ to C₁₈ alkyl sarcosine is to be used, preferably at least some of the acidic carboxyl groups are neutralized with, for example, sodium hydroxide, to render the surfactant water dispersible.

Particularly preferred non-ionic or amphoteric or sarcosine anionic surfactants include one or more of Cocomidopropyl betaine, Polyethyleneglycol lauryl ether (e.g., Brij 35), Poloxamer 188, Polysorbate 80, PEG-7 Glyceryl Cocoate, PEG-7 oleamide, 2-[4-(2,4,4-trimethylpentan-2-yl)phenoxy]ethanol (e.g., Triton X-100), Polysorbate 20, Poloxamer 407, Cocomidopropylamine oxide and lauramidopropyl betaine.

The antimicrobial composition comprises one or more non-ionic or amphoteric or sarcosine anionic surfactants at a concentration of about 0.05% to about 2% w/v, preferably at a concentration of about 0.1% to about 1% w/v. The composition may comprise one or more non-ionic or amphoteric or sarcosine anionic surfactants at a concentration of about 0.4% w/v.

Optionally, the antimicrobial composition additionally comprises a chelating agent, which may be present at a concentration of from about 0.01% to about 0.2% w/v, preferably about 0.01% to about 0.1% w/v. The composition may comprise a chelating agent at about 0.05% w/v.

The chelating agent may be selected from one or more of disodium EDTA, Trisodium EDTA and tetrasodium EDTA.

Optionally, the antimicrobial composition additionally comprises a polymeric biguanide. Suitable polymeric biguanides include one or more of chlorhexidine and polyhexamethylene biguanide.

Preferably the antimicrobial composition has a pH of about 4.5 to about 8.5 or about 5 to about 7 or about 5.5 to about 6.5. The antimicrobial composition may have a pH of about 5.5 or about 6.5. The pH of the composition can be adjusted in the range of from about 4.5 to about 8.5 using an appropriate organic or inorganic acid such as citric acid, acetic acid, hydrochloric acid, phosphoric acid, sorbic acid or an organic or inorganic base such as ammonium hydroxide, sodium hydroxide, potassium hydroxide, ethyl amine, dimethyl amine, triethyl amine, ethanol amine, diethanol amine and triethanol amine.

Preferably the antimicrobial composition does not include a lipid. More preferably the formulation antimicrobial composition does not include a phospholipid.

Examples of antimicrobial compositions of the invention include:

An antimicrobial composition comprising:
a quarternary ammonium silane, such as Dimethyloctadecyl[3-(trimethoxysilyl)propyl]ammonium chloride, at a concentration of from about 0.01% to about 0.4% or about 0.5% w/v;
one or more quarternary ammonium compounds such as Benzalkonium chloride and/or Didecyldimethylammonium chloride at a concentration of from about 0.01% to about 0.4% or about 0.5% w/v;
an optional biguanide such as chlorohexidine or polyhexanide;
a chelating agent such as disodium EDTA or tetrasodium EDTA at a concentration from about 0.01% to 0.2% w/v; and
a nonionic or amphoteric surfactant such as Cocomidopropyl betaine, Brij 35, Pluronic F68, Tween 80 at a concentration of from about 0.05% to about 1% w/v;
the composition having a pH of from about 4.5 to about 8.5.

An antimicrobial composition comprising:
about 0.1% w/v Dimethyloctadecyl[3-(trimethoxysilyl)propyl]ammonium chloride;
about 0.1% w/v Benzalkonium chloride;
about 0.1% w/v Didecyldimethylammonium chloride;
about 0.1% w/v chlorohexidane digluconate;
about 0.18% or about 0.36% or about 0.72% or about 1.08% w/v Cocomidopropyl betaine; and
about 0.05% tetrasodium EDTA;
the composition having a pH of from about 5 to about 7.

An antimicrobial composition comprising:
about 0.1% w/v Dimethyloctadecyl[3-(trimethoxysilyl)propyl]ammonium chloride;
about 0.1% w/v Benzalkonium chloride;
about 0.1% w/v Didecyldimethylammonium chloride; and
about 0.4% w/v Cocomidopropyl betaine.

An antimicrobial composition comprising:
about 0.1% w/v Dimethyloctadecyl[3-(trimethoxysilyl)propyl]ammonium chloride;
about 0.1% w/v Benzalkonium chloride;
about 0.1% w/v Didecyldimethylammonium chloride;
about 0.05% w/v disodium EDTA; and
about 0.4% w/v Cocomidopropyl betaine.

The antimicrobial composition may further comprise one or more pharmaceutically acceptable carriers and/or excipients, such as diluents, adjuvants, excipients, vehicles, fillers, binders, disintegrating agents, wetting agents, emulsifying agents, suspending agents, perfuming agents, buffers, dispersants, thickeners, solubilising agents, lubricating agents and dispersing agents, depending on the nature of the mode of administration and dosage forms.

The antimicrobial composition is preferably in the form of a liquid preparation, such as a gel, lotion, sprays or/and solution. Preferably the composition is in the form of a solution. The composition is preferably formulated for topical administration, such as in the form of a wound irrigation solution.

The antimicrobial composition can be used in therapy, such as for treating and/or preventing a skin and/or wound infection. The infection may be a bacterial infection, which may comprise a biofilm.

The antimicrobial composition may be an antibacterial composition, an antifungal composition, an antiparasitic composition or an antibiofilm composition. The composition may be used to treat or prevent bacterial infections such as Gram-negative or Gram-positive bacterial infections, fungal infections, parasitic infections, or biofilms as appropriate.

Gram-positive bacteria include, for example, Streptococci, such as S. viridans, Staphylococci, such as S. aureus, and Bacillus, such as B. subtilis, B. anthracis and B. cereus.

Gram-negative bacteria include, for example, E. coli, Pseudomonas, such as P. aeruginosa, and Klebsiella, such as K. pneumonia, K. aerogenes and K. oxytoca.

Compositions of the present invention may be for use in treating or preventing a fungal infection. The fungal infection may be a Candida infection, for example, an infection with C. albicans, C. parapsilosis or C. tropicalis, or a combination thereof.

The wound may be an open wound and may be acute or chronic. Antimicrobial compositions as described herein can be used for the cleansing, moisturizing and decontamination of acute wounds, chronic wounds, thermal wounds, chemical, radiation-induced wounds, and superficial burns. The compositions can also be used for intraoperative wound cleansing and irrigation.

The present invention also provides a method of treating or preventing a skin and/or wound infection on a patient, the method comprising administering an antimicrobial composition as described herein to the skin and/or to the wound.

Administering the antimicrobial composition to the skin and/or to the wound typically refers to contacting the skin and/or wound with the antimicrobial composition. The antimicrobial composition may be placed in contact with the skin and/or wound for about 1 minute, or about 5 minutes or about 10 minutes.

In certain embodiments, the patient is a human, a primate, bovine, ovine, equine, porcine, avian, rodent (such as mouse or rat), feline, or canine. Preferably, the patient is a human. The patient can also be production animals such as cattle, oxen, deer, goats, sheep and pigs, working and sporting animals such as dogs, horses and ponies, companion animals such as dogs and cats, and laboratory animals such as rabbits, rats, mice, hamsters, gerbils or guinea pigs.

The present invention additional provides a method of making an antimicrobial composition as described herein, the method comprising (a) combining: (i) a water soluble organosilane at a concentration of about 0.01% to about 0.4% or about 0.5% w/v; (ii) one or more quarternary ammonium compounds at concentration of about 0.01% to 0.5% w/v; (iii) a non-ionic or amphoteric surfactant at a concentration of about 0.05% to about 1% w/v; and (iv) water to form a solution; and (b) adjusting the pH of the solution to about 5.5 to about 8.5.

### BRIEF DESCRIPTION OF THE DRAWINGS

The invention will now be described in detail, by way of example only, with reference to the figures.

FIG. 1—(A) MBEC (24 h) of Pseudomonas aeruginosa ATCC 15442 when treated with either Hybrisan Technology or Prontosan® at contact times of 1- and 5-min. (B) MBEC (24 h) of Staphylococcus aureus ATCC 6538 when treated with either Hybrisan Technology or Prontosan® at contact times of 1- and 5-min. Error bars represent standard deviation, * represents a significant log reduction (p<0.05) in comparison to the control or between time points (N=3).

FIG. 2—Established static (24 h) biofilms of Pseudomonas aeruginosa ATCC 15442 when treated with either Hybrisan Technology or Prontosan® at contact times of 1- and 5-min. Error bars represent standard deviation, * represents a significant log reduction (p<0.05) in comparison to the control or between time points (N=3).

FIG. 3—Impact of either Hybrisan Technology or Prontosan® after treatment (5 min) on the regrowth of static (24 h) biofilms of Pseudomonas aeruginosa ATCC 15442 when treated with either Hybrisan Technology or Prontosan®. (N=3).

FIG. 4—24 hour biofilms when treated with Hybrisan Technology or Prontosan® of (A) Pseudomonas aeruginosa ATCC 15442 grown in the CDC bioreactor model (ASTM E2871-13) at contact times of 1, 5 and 10 mins (B) Staphylococcus aureus ATCC 29213 grown in the CDC bioreactor model (ASTM E2871-13) at contact times of 1, 5 and 10 mins (C) Candida albicans ATCC 10231 grown in the CDC bioreactor model (ASTM E2871-13) at contact times of 1, 5 and 10 mins (D) Pseudomonas aeruginosa ATCC 15442 grown in the drip flow reactor (ASTM E2647-13) at a contact time of 5 mins (E) Staphylococcus aureus ATCC 29213 grown in the drip flow reactor (ASTM E2647-13) at a contact time of 5 mins.

FIG. 5—Cell viability (%) of mouse fibroblast cells, L929, when treated with Hybrisan Technology and Prontosan® for 5 mins, 10 mins and 24 hours when tested according to ISO 10993:5-2009).

FIG. 6—(A) MBEC (24 h) of Pseudomonas aeruginosa ATCC 15442 when treated with either v9 or Prontosan® at contact times of 1- and 5-min. (B) MBEC (24 h) of Staphylococcus aureus ATCC 6538 when treated with either v9 or Prontosan® at contact times of 1- and 5-min. Error bars represent standard deviation, * represents a significant log reduction (p<0.05) in comparison to the control or between time points (N=3).

FIG. 7—Established static (24 h) biofilms of Pseudomonas aeruginosa ATCC 15442 when treated with either v9 or Prontosan® at contact times of 1 and 5-min. Error bars represent standard deviation, * represents a significant log reduction (p<0.05) in comparison to the control or between time points (N=3).

FIG. 8—Impact of either v9 or Prontosan® after treatment (5 min) on the regrowth of static (24 h) biofilms of Pseudomonas aeruginosa ATCC 15442 when treated with either Hybrisan Technology or Prontosan®. (N=3).

### EXAMPLES

### Example 1

An exemplary formulation of the invention, referred to herein as
“Hybrisan Technology” can be found in Table 1 below:

| Ingredients | CAS | Quantity (%) |
|---|---|---|
| Alkyl Dimethyl Benzyl Ammonium | 68424-85-1 | 0.1 |
| Chloride (Benzalkonium Chloride) | | |
| N,N-Didecyl-N,N- | 7173-51-5 | 0.1 |
| dimethylammoniumchloride | | |
| (Didecyldimonium chloride) | | |
| Dimethyloctadecyl[3- | 27668-52-6 | 0.1 |
| (trimethoxysilyl)propyl]ammonium | | |
| chloride | | |
| Cocamidopropyl Betaine | 97862-59-4 | 0.36 |
| Disodium EDTA | 6381-92-6 | 0.05 |
| Other ingredients (carriers for raw | | 0.3 |
| materials) | | |
| Water | | 98.99 |
| pH | | 6.5 |

### Biocompatibility Testing

### Cytotoxicity Testing

Cytotoxicity tests refer to in-vitro assays to assess the ability of the test article to cause cell death or to inhibit cell growth. Tests for in vitro cytotoxicity specify procedures for testing liquids by direct contact, and Hybrisan Technology has been assessed for cell morphology changes both qualitatively and quantitatively. Hybrisan Technology has been tested by exposure to the cell culture medium following ISO standard, Biological evaluation of medical devices—Part 5: Tests for in vitro cytotoxicity (ISO 10993-5:2009) and compared to a non-treatment control, PBS and a positive control of a commercially available wound irrigation solution, Prontosan®. In addition to this, two shorter time points (5- and 10-min) were also tested, as these are closer to the clinical application of Hybrisan Technology. Testing was conducted at an independent laboratory at 5D Health Protection Group Ltd.

### Cytotoxicity Testing—Qualitative Evaluation

Mouse fibroblast cells, L929, were used to evaluate cell viability and proliferation after treatment with Hybrisan Technology, Prontosan®, or PBS compared to a no treatment control and analysed by microscopy. Following all treatment, a reduction in size was seen for cell morphology. An increase in cell lysis, vacuolization and floating cells was seen when treated with Prontosan®. PBS did not affect cell morphology, but more cell detachment was seen after 24 h. Overall the qualitative assessment concluded that Hybrisan Technology affects cell morphology less than Prontosan®.

### Cytotoxicity Testing—Quantitative Evaluation

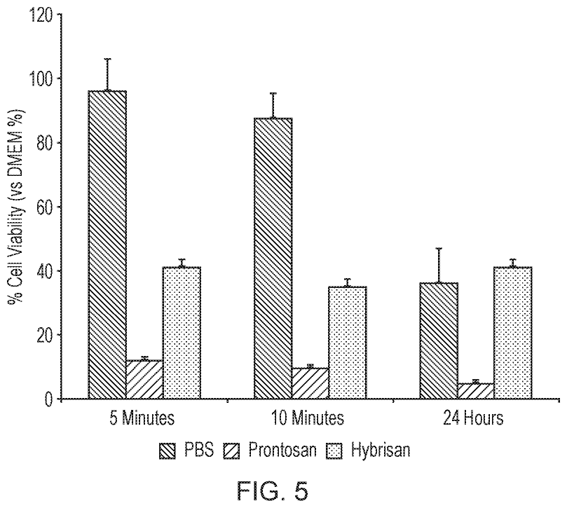

Quantitative cytotoxicity testing used CyQUANT® viability dye to test the wound irrigation solutions at neat concentration. Percentage viability is shown in Table 2 and FIG. 5 (a higher percentage number shows that more cells are viable). As expected, Hybrisan Technology is cytotoxic against L929 cells. Hybrisan Technology was less cytotoxic than Prontosan® at all time points.

TABLE 2
Percentage viability of Hybrisan Technology, Prontosan ® and
PBS, when compared to a no treatment control (N = 5).

| | 100% | | |
|---|---|---|---|
| | 5 min | 10 min | 24 h |
| Hybrisan | 41 | 35 | 17 |
| Prontosan ® | 12 | 10 | 5 |
| PBS | 96 | 87 | 36 |

### Antimicrobial Testing

Hybrisan Technology has been systematically tested both in-house and independently. The purpose of the testing has been to evaluate the antimicrobial and antibiofilm efficacy of Hybrisan Technology against Pseudomonas aeruginosa, Staphylococcus aureus and Candida albicans, the etiological agents for chronic wound infections.

In house testing has revealed that Hybrisan Technology is an effective antimicrobial agent. This was confirmed by antimicrobial susceptibility testing following ISO 20776-1:2019 and antibiofilm efficacy testing following ASTM E2799.

### Susceptibility Testing

Minimal inhibitory concentration (MIC) and Minimum Bactericidal Concentration (MBC) of Hybrisan Technology were determined for both Pseudomonas aeruginosa and for Staphylococcus aureus using a method recommended by the Clinical and Laboratory Standards Institute (CLSI), the European Committee on Antimicrobial Susceptibility Testing (EUCAST; www.Eucast.org) and ISO 20776-1:2019. A MIC of 23.4 ppm and 0.72 ppm for Pseudomonas aeruginosa and for Staphylococcus aureus and an MBC of 46.89 ppm and 4.39 ppm for Pseudomonas aeruginosa and for Staphylococcus aureus were achieved by Hybrisan Technology.

TABLE 3
MIC and MBC results of Hybrisan Technology
using EUCAST test methods.

| Microorganisms | MIC (ppm) | MBC (ppm) |
|---|---|---|
| Pseudomonas aeruginosa ATCC | 23.4 | 46.89 |
| 15442 | | |
| Staphylococcus aureus ATCC | 0.72 | 4.39 |
| 6538 | | |

### Minimum Biofilm Eradication Concentration (MBEC) (ASTM E2799)

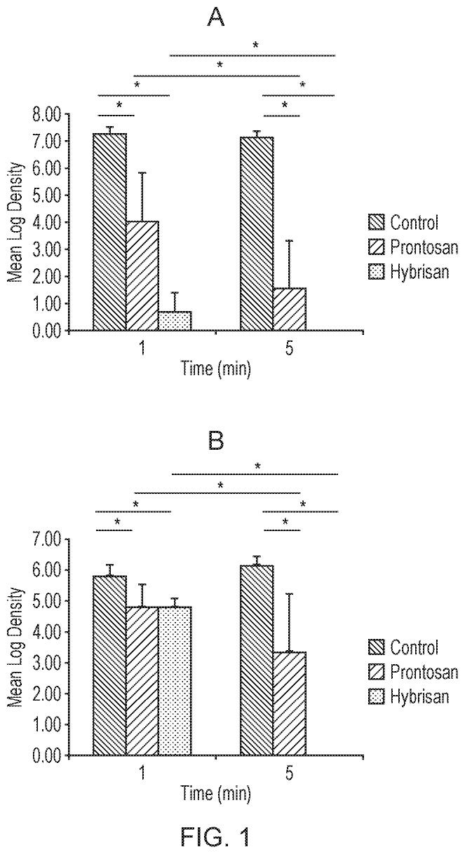

Further studies in-house confirmed that Hybrisan Technology is an effective antibiofilm product using the MBEC ASTM E2799 protocol with wound microorganisms. Pseudomonas aeruginosa ATCC 15442 biofilms when grown for 24 h and treated with Hybrisan Technology produced a 6.6 and a 7.2 log reduction while Prontosan® indicated a 3.2 and 5.6 log reduction at 1 and 5 min respectively (FIG. 1A). These were both significant reductions in biofilm biomass. Staphylococcus aureus ATCC 6538 biofilms when grown for 24 h and treated with Hybrisan Technology produced a 1.1 and a 6.3 log reduction while Prontosan indicated a 1.1 and 1.9 log reduction at 1 and 5 min respectively (FIG. 1B). Efficacy improved depending on contact time and significant reductions in biofilm biomass were seen for both products. However, Hybrisan Technology consistently outperformed Prontosan®.

### Impact of Hybrisan Technology on Static Biofilms; Established (24 h), and Regrowth

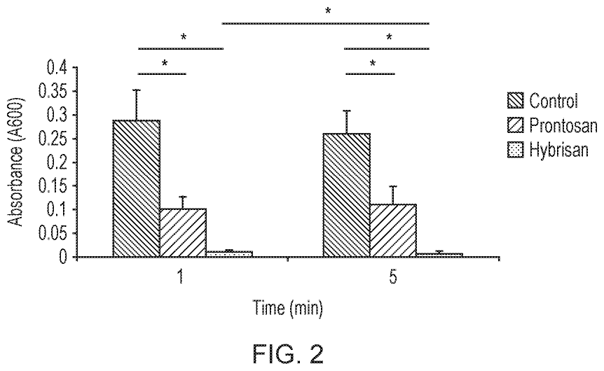

Established biofilms of Pseudomonas aeruginosa ATCC 15442 were grown for 24 h and treated with Hybrisan technology or Prontosan® for either 1 or 5 min and compared to a no-treatment control. A significant reduction was seen for both Hybrisan Technology and Prontosan®. At both 1 and 5 min a greater reduction was seen for Hybrisan Technology (94 and 96%) than for Prontosan® (59 and 57%) respectively (FIG. 2).

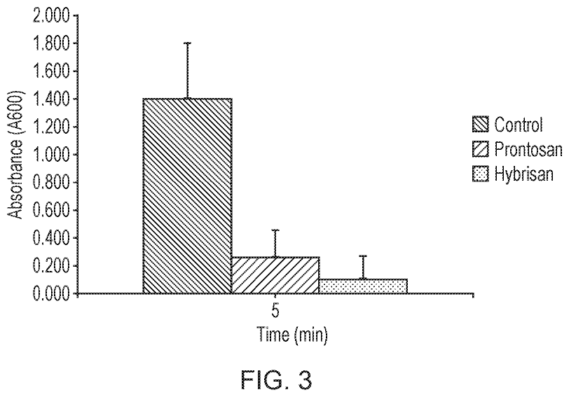

A further study was conducted where biofilms (24 h) were grown and treated with either Hybrisan Technology or Prontosan® for 5 min and then incubated for a further 24 h in the presence of growth media. This has been conducted to evaluate the latent efficacy of either treatment. A significant reduction of growth was seen for both Hybrisan Technology and Prontosan®, although there was less regrowth seen for Hybrisan Technology, when compared to the no treatment control. The no treatment control growth was 50% of what is normally seen in a 24 h static biofilm. Regrowth following treatment with Hybrisan Technology was minimal (FIG. 3).

### Independent Testing Antimicrobial Testing

5D Health Protection Group, has proven that Hybrisan Technology has greater antibiofilm efficacy when tested against similar commercially available products e.g., Prontosan®.

CDC Bioreactor Model—P. aeruginosa, S. Aureus and C. albicans

Using the Centers for Disease Control (CDC) biofilm reactor following ASTM E2871-13: an in vitro method used to produce robust and clinically similar biofilms. This well-respected method has shown that Hybrisan Technology works faster and eradicates more microbial biofilms than Prontosan®, over clinically significant time periods (1 min, 5 min, 10 min) for all strains tested P. aeruginosa, S. aureus and C. albicans (FIGS. 5A, 5B and 5C).

Hybrisan Technology showed completed eradication of P. aeruginosa after 5 min while only a 2-log reduction was seen for Prontosan® (FIG. 5A). Again, complete eradication of S. aureus was seen after 5 min while only a 2.3 log reduction was seen for Prontosan® (FIG. 5B). As expected, Hybrisan Technology and Prontosan® were not able completely eradicate C. albicans biofilms after 10 min (FIG. 5C), however Hybrisan Technology consistently performed better against Prontosan® and showed greater antibiofilm efficacy.

Drip Flow Biofilm Reactor Model—P. aeruginosa and S. aureus

Using the drip flow biofilm reactor following ASTM E2647-13 (standard test method for quantification of Pseudomonas aeruginosa biofilm grown with using drip flow biofilm reactor with low shear and continuous flow). This validated model has again shown that Hybrisan Technology works faster and eradicates more microbial biofilms than Prontosan® over a clinically significant time period (5 min) for both strains tested P. aeruginosa and S. aureus (FIGS. 5D and 5E).

Hybrisan Technology showed a 4.26 log reduction of P. aeruginosa after a 5-min contact time while only a 3.62 log reduction was seen for Prontosan® (FIG. 5D). Complete eradication of S. aureus was seen after 5 min while only a 3.8 log reduction was seen for Prontosan® (FIG. 5E).

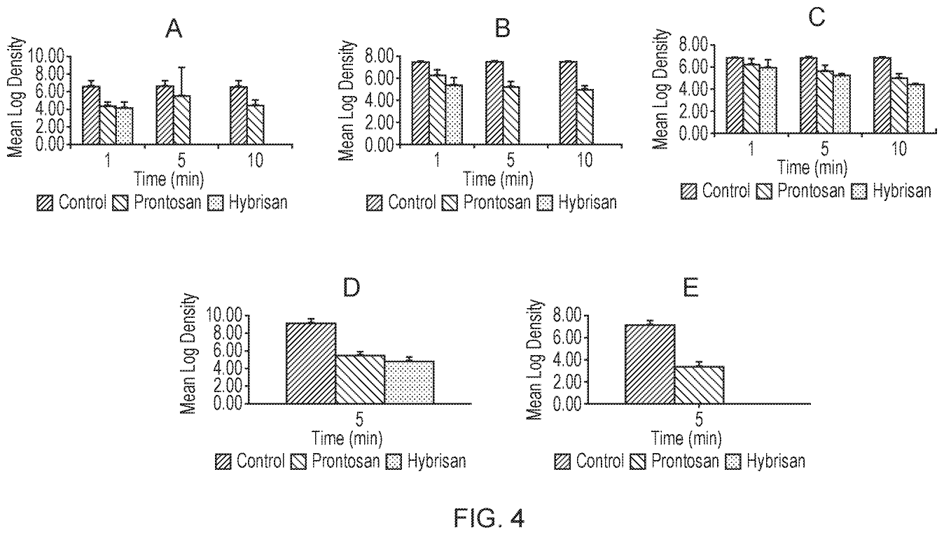

TABLE 4
CDC biofilm counts and log reduction (Woundsan = Hybrisan Technology)

| CDC biofilm | | CFU/ml at Time points (min) | | | | log reduction at Time points (min) | | | |
|---|---|---|---|---|---|---|---|---|---|
| Treatment | Microorganism | 1 | 5 | 10 | 15 | 1 | 5 | 10 | 15 |
| Control | P. aeruginosa | 3.85E+06 | 3.85E+06 | 3.85E+06 | 3.85E+06 | — | — | — | — |
| Woundsan | ATCC 15442 | 1.41E+04 | 0.00E+00 | 0.00E+00 | 0.00E+00 | 2.44 | 6.59 | 6.59 | 6.59 |
| Prontosan ® | (FIG. 4A) | 2.20E+04 | 3.20E+05 | 2.87E+04 | 0.00E+00 | 2.25 | 1.08 | 2.13 | 6.59 |
| Control | S. aureus | 3.40E+07 | 3.40E+07 | 3.40E+07 | — | — | — | — | — |
| Woundsan | ATCC 29213 | 2.22E+05 | 0.00E+00 | 0.00E+00 | — | 2.18 | 7.53 | 7.53 | — |
| Prontosan ® | (FIG. 4B) | 1.98E+06 | 1.69E+05 | 9.80E+04 | — | 1.23 | 2.30 | 2.54 | — |
| Control | C. albicans | 7.70E+06 | 7.70E+06 | 7.70E+06 | — | — | — | — | — |
| Woundsan | ATCC 10231 | 9.17E+05 | 2.13E+05 | 2.73E+04 | — | 0.92 | 1.56 | 2.45 | — |
| Prontosan ® | (FIG. 4C) | 1.70E+06 | 4.63E+05 | 1.14E+05 | — | 0.66 | 1.22 | 1.83 | — |

TABLE 5
Drip flow counts and log reduction -
5 min (Woundsan = Hybrisan Technology)

| Treatment | Microorganism | CFU/ml | Log reduction |
|---|---|---|---|
| Control | P. aeruginosa | 1.45E+09 | — |
| Woundsan | ATCC 15442 | 7.90E+04 | 4.26 |
| Prontosan ® | (FIG. 4D) | 3.45E+05 | 3.62 |
| Control | S. aureus ATCC | 2.03E+07 | — |
| Woundsan | 29213 | 0.00E+00 | 7.31 |
| Prontosan ® | (FIG. 4E) | 3.47E+03 | 3.8 |

These data have shown unequivocally that Hybrisan Technology is an effective antimicrobial and antibiofilm product that performs better than the market leader. Our expert independent testing facility suggest that Hybrisan Technology could be used in the clinic to treat biofilms, such as those that have formed in chronic wounds.

### Example 2

### Methods

Formulations were prepared according to Tables 6-13 and tested for stability and minimal biofilm eradication concentration (MBEC).

### MBEC:

The MBEC model was adapted from ASTM E2799. In brief, an overnight culture of P. aeruginosa or S. aureus was prepared in Mueller Hinton Broth (MHB) and diluted to ˜1×10⁵ CFU/ml. The wells of a 96-well plate (Nunc, Thermo Fisher, UK) were inoculated with 100 μl of the test inoculum. A 96-peg lid (Nunc, Thermo Fisher, UK) was added to the plate and incubated for 24 hours at 37° C. in a humidified container shaking at 110 rpm to form biofilms. After incubation, biofilms were washed in sterile 0.85% Sodium Chloride (Thermo Fisher, UK) solution in deionised water. Following rinsing, the lids were transferred to a new 96-well plate containing 150 μl of test solution. Prontosan® (B. Braun, Germany) was used as a positive control and 0.85% Sodium Chloride solution in deionised water was used as a negative control. Pegs were treated for 5 minutes. After treatment pegs were removed and placed in wells containing a suitable neutralising media and placed in an ultrasonic bath for 30 minutes at full power. Each well was serially diluted and total viable counts (TVCs) were determined. Log reduction was calculated using the following formula:

Log Reduction=log₁₀(A/B)

Where A is the number of viable organisms before treatment and B is the number of viable organisms after treatment.

### Stability:

For initial stability testing a 100 ml sample of the composition was prepared and 10 ml was transferred into each of 3 15 ml tubes. These tubes were placed at 4° C., 20° C. and 37° C. These samples were then monitored for stability and graded weekly for a period of 4 weeks. 1=clear, 2=hazy, 3=precipitated, 4=hazy and precipitated.

Accelerated aging was performed on selected samples to ensure their long-term stability. For this a protocol was established based on ASTM F1980. In brief, the required test period was determined using the Arrhenius equation:

$$\\ Accelerated Ageing Time = \frac{Desired Real Time Period}{Q 10 [ \frac{TAA - TRT}{10} ]}$$

Test Temperature (TAA—° C.)
Ambient Storage Temperature (TRT—° C.)
Q10 (Reaction Rate Factor)
Real Time Equivalent (days)

A sample of 100 ml was prepared and transferred to a 50 ml tube. The weight of the tube was reported to ensure no significant evaporation of the product occurs over the test period. This tube was then placed in an incubator at the defined test temperature and initially checked daily for changes for the first 4 weeks. After 4 weeks the tube was checked weekly until the end of the test. At the end of the test the tube was allowed to return to room temperature, weighed to ensure no significant evaporation had occurred and tested to ensure it had maintained efficacy over the test period.

| Formulation | v11.1.1 | v11.1.2 | v11.1.3 | v11.2.1 | v11.2.2 | v11.2.3 | v11.3.1 | v11.3.2 | v11.3.3 | v11.4.1 | v11.4.2 | v11.4.3 |
|---|---|---|---|---|---|---|---|---|---|---|---|---|
| BAC | 0.1 | 0.1 | 0.1 | 0.1 | 0.1 | 0.1 | 0.1 | 0.1 | 0.1 | 0.1 | 0.1 | 0.1 |
| DDAC | 0.1 | 0.1 | 0.1 | 0.1 | 0.1 | 0.1 | 0.1 | 0.1 | 0.1 | 0.1 | 0.1 | 0.1 |
| Organosilane | 0.1 | 0.1 | 0.1 | 0.1 | 0.1 | 0.1 | 0.1 | 0.1 | 0.1 | 0.1 | 0.1 | 0.1 |
| Chlorohexidane | 0.1 | 0.1 | 0.1 | 0.1 | 0.1 | 0.1 | 0.1 | 0.1 | 0.1 | 0.1 | 0.1 | 0.1 |
| Digluconate | | | | | | | | | | | | |
| Tween | — | — | — | — | — | — | — | — | — | — | — | — |
| Brij35 | — | — | — | — | — | — | — | — | — | — | — | — |
| Pluronic F68 | — | — | — | — | — | — | — | — | — | — | — | — |
| Coco-betaine | 0.18 | 0.18 | 0.18 | 0.36 | 0.36 | 0.36 | 0.72 | 0.72 | 0.72 | 1.08 | 1.08 | 1.08 |
| Tetrasodium | 0.05 | 0.05 | 0.05 | 0.05 | 0.05 | 0.05 | 0.05 | 0.05 | 0.05 | 0.05 | 0.05 | 0.05 |
| EDTA | | | | | | | | | | | | |
| Disodium EDTA | — | — | — | — | — | — | — | — | — | — | — | — |
| Glycerol | — | — | — | — | — | — | — | — | — | — | — | — |
| Dipropylene | — | — | — | — | — | — | — | — | — | — | — | — |
| glycol | | | | | | | | | | | | |
| pH | 5.5 | 6 | 6.5 | 5.5 | 6 | 6.5 | 5.5 | 6 | 6.5 | 5.5 | 6 | 6.5 |
| Stability (1-4) | 1 | 1 | 1 | 1 | 1 | 1 | 1 | 1 | 1 | 1 | 1 | 1 |
| Log Reduction | 7.5 | — | 7.5 | 7.5 | — | 7.5 | 3.09 | — | 7.5 | 2.8 | — | 7.5 |
| (MBEC) | | | | | | | | | | | | |

Table 6: shows the impact of cocamidopropyl betaine and pH on stability and minimal biofilm eradication concentration (MBEC).
(Amounts are % w/v; — shows that MBEC was not tested).
v11.3.1 and v11.4.1 showed good antimicrobial efficacy but were significantly less effective against biofilms.

| Formulation | v12.2 | v12.3 | v13.1 | v13.2 | v13.3 |
|---|---|---|---|---|---|
| BAC | 0.1 | 0.1 | — | 0.1 | — |
| DDAC | 0.1 | 0.1 | 0.1 | — | — |
| Organosilane | 0.25 | 0.5 | 0.1 | 0.1 | 0.1 |
| Chlorohexidane | 0.1 | 0.1 | 0.1 | 0.1 | 0.1 |
| Digluconate | | | | | |
| Tween | — | — | — | — | — |
| Brij35 | — | — | — | — | — |
| Pluronic F68 | — | — | — | — | — |
| Coco-betaine | 0.36 | 0.36 | 0.36 | 0.36 | 0.36 |
| Tetrasodium | — | — | — | — | — |
| EDTA | | | | | |
| Disodium EDTA | 0.05 | 0.05 | 0.05 | 0.05 | 0.05 |
| Glycerol | — | — | — | — | — |
| Dipropylene | — | — | — | — | — |
| glycol | | | | | |
| pH | 6.5 | 6.5 | 6.5 | 6.5 | 6.5 |
| Stability (1-4) | 1 | 2 | 3 | 3 | 3 |
| Log Reduction | — | 7.5 | 5.22 | 4.5 | 3.15 |
| (MBEC) | | | | | |

Table 7 shows the effect of increasing concentrations of organosilane (v12.2-v12.3), no BAC (v13.1), no DAC (v13.2) and no BAC and DAC (v13.3) on stability and minimal biofilm eradication concentration (MBEC). (Amounts are % w/v; — shows that MBEC was not tested).

| Formulation | v13.4 | V13.4.1 | v13.5 | V13.6.1 | V13.7.1 |
|---|---|---|---|---|---|
| BAC | 0.1 | 0.2 | 0.1 | 0.1 | 0.1 |
| DDAC | 0.1 | 0.2 | 0.1 | 0.1 | 0.1 |
| Organosilane | 0.1 | 0.2 | 0.1 | 0.1 | 0.1 |
| Chlorohexidane | — | — | 0.1 | — | — |
| Digluconate | | | | | |
| Tween | — | — | — | — | — |
| Brij35 | — | — | — | — | — |
| Pluronic F68 | — | — | — | — | — |
| Coco-betaine | 0.36 | 0.72 | 0.36 | 0.36 | 0.18 |
| Tetrasodium | — | — | — | — | — |
| EDTA | | | | | |
| Disodium EDTA | 0.05 | 0.1 | 0 | — | — |
| Glycerol | — | — | — | — | — |
| Dipropylene | — | — | — | — | — |
| glycol | | | | | |
| pH | 6.5 | 6.5 | 6.5 | 6.5 | 6.5 |
| Stability (1-4) | 1 | 2 | 1 | 1 | 1 |
| Log Reduction | 7.5 | 7.5 | 7.5 | — | — |
| (MBEC) | | | | | |

Table 8 shows the effect of no chlorohexidane digluconate (v13.4 and v13.4.1), no EDTA (v13.5), no chlorohexidane digluconate and no EDTA (v13.6.1) and no chlorohexidane digluconate and no EDTA with reduced coco-betaine (v13.7.1). (Amounts are % w/v; — shows that MBEC was not tested).

| Formulation | v8.8 | v8.9 | v8.2 |
|---|---|---|---|
| BAC | 0.1 | 0.1 | 0.1 |
| DDAC | 0.1 | 0.1 | 0.1 |
| Organosilane | 0.1 | 0.1 | 0.1 |
| Chlorohexidane | 0.1 | 0.1 | 0.1 |
| Digluconate | | | |
| Tween | — | — | — |
| Brij35 | — | — | — |
| Pluronic F68 | 0.1 | 0.5 | 1 |
| Coco-betaine | — | — | — |
| Tetrasodium | — | — | — |
| EDTA | | | |
| Disodium EDTA | 0.05 | 0.05 | 0.05 |
| Glycerol | 10 | 20 | 50 |
| Dipropylene | — | — | — |
| glycol | | | |
| pH | 6.5 | 6.5 | 6.5 |
| Stability (1-4) | 4 | 4 | 4 |
| Log Reduction | — | — | — |
| (MBEC) | | | |

Table 9 shows the effect of increasing F-68 concentration (0.1%; v8.8) (0.5%; v8.9) (1%; v8.2) on stability. (Amounts are % w/v; — shows that MBEC was not tested).

| Formulation | v14.1 | v14.2 | v14.3 | v14.4 | v14.5 |
|---|---|---|---|---|---|
| BAC | 0.1 | 0.1 | 0.1 | 0.1 | 0.1 |
| DDAC | 0.1 | 0.1 | 0.1 | 0.1 | 0.1 |
| Organosilane | 0.1 | 0.1 | 0.1 | 0.1 | 0.1 |
| Chlorohexidane | 0.1 | 0.1 | 0.1 | 0.1 | 0.1 |
| Digluconate | | | | | |
| Tween | — | — | — | — | — |
| Brij35 | — | — | — | — | — |
| Pluronic F68 | — | — | — | — | — |
| Coco-betaine | 0.36 | 0.36 | 0.36 | 0.36 | 0.36 |
| Tetrasodium | — | — | — | — | — |
| EDTA | | | | | |
| Disodium EDTA | 0.05 | 0.05 | 0.05 | 0.05 | 0.05 |
| Glycerol | 10 | 20 | 40 | 50 | 60 |
| Dipropylene | — | — | — | — | — |
| glycol | | | | | |
| pH | 6.5 | 6.5 | 6.5 | 6.5 | 6.5 |
| Stability (1-4) | 1 | 1 | 1 | 2 | 4 |
| Log Reduction | 7.5 | — | — | — | — |
| (MBEC) | | | | | |

Table 10 shows the effect of increasing concentrations of glycerol on stability. (Amounts are % w/v; — shows that MBEC was not tested).

| Formulation | 7.3 | 7.1 | 7.2 |
|---|---|---|---|
| BAC | 0.1 | 0.1 | 0.1 |
| DDAC | 0.1 | 0.1 | 0.1 |
| Organosilane | 0.1 | 0.1 | 0.1 |
| Chlorohexidane | 0.1 | 0.1 | 0.1 |
| Digluconate | | | |
| Tween | — | — | — |
| Brij35 | 0.1 | 1 | 2 |
| Pluronic F68 | — | — | — |
| Coco-betaine | — | — | — |
| Tetrasodium | — | — | — |
| EDTA | | | |
| Disodium | — | — | — |
| EDTA | | | |
| Glycerol | 5 | 5 | 5 |
| Dipropylene | — | — | — |
| glycol | | | |
| pH | 6.5 | 6.5 | 6.5 |
| Stability (1-4) | 2 | 1 | 1 |
| Log | — | 7.5 | 7.5 |
| Reduction | | | |
| (MBEC) | | | |

Table 11 shows the effect of Brij35 concentration (0.1%; v7.3) (1%; v7.1) (2%; v7.2) on stability and minimal biofilm eradication concentration (MBEC). (Amounts are % w/v; — shows that MBEC was not tested).

| Formulation | v15.1 | v15.2 | v15.3 | v15.4 | v15.5 |
|---|---|---|---|---|---|
| BAC | 0.1 | 0.1 | 0.1 | 0.1 | 0.1 |
| DDAC | 0.1 | 0.1 | 0.1 | 0.1 | 0.1 |
| Organosilane | 0.1 | 0.1 | 0.1 | 0.1 | 0.1 |
| Chlorohexidane | 0.1 | 0.1 | 0.1 | 0.1 | 0.1 |
| Digluconate | | | | | |
| Tween | — | — | — | — | — |
| Brij35 | — | — | — | — | — |
| Pluronic F68 | — | — | — | — | — |
| Coco-betaine | 0.36 | 0.36 | 0.36 | 0.36 | 0.36 |
| Tetrasodium | — | — | — | — | — |
| EDTA | | | | | |
| Disodium | 0.05 | 0.05 | 0.05 | 0.05 | 0.05 |
| EDTA | | | | | |
| Glycerol | — | — | — | — | — |
| Dipropylene | 10 | 20 | 40 | 50 | 60 |
| glycol | | | | | |
| pH | 6.5 | 6.5 | 6.5 | 6.5 | 6.5 |
| Stability (1-4) | 4 | 4 | 4 | 4 | 4 |
| Log Reduction | 7.5 | — | — | — | — |
| (MBEC) | | | | | |

Table 12 shows the effect of dipropylene glycol concentration (10%; v15.1) (20%; v15.2) (40%; v15.3) (50%; v15.4) (60%; v15.5) on stability. (Amounts are % w/v; — shows that MBEC was not tested).

| Formulation | v15.6 | v15.7 | v15.8 | v14.6 | v14.7 | v14.8 |
|---|---|---|---|---|---|---|
| BAC | 0.2 | 0.2 | 0.2 | 0.2 | 0.2 | 0.2 |
| DDAC | 0.2 | 0.2 | 0.2 | 0.2 | 0.2 | 0.2 |
| Organosilane | 0.1 | 0.1 | 0.1 | 0.1 | 0.1 | 0.1 |
| Chlorohexidane | 0.1 | 0.1 | 0.1 | 0.1 | 0.1 | 0.1 |
| Digluconate | | | | | | |
| Tween | — | — | — | — | — | — |
| Brij35 | — | — | — | — | — | — |
| Pluronic F68 | — | — | — | — | — | — |
| Coco-betaine | 0.36 | 0.36 | 0.36 | 0.36 | 0.36 | 0.36 |
| Tetrasodium | — | — | — | — | — | — |
| EDTA | | | | | | |
| Disodium EDTA | 0.05 | 0.05 | 0.05 | 0.05 | 0.05 | 0.05 |
| Glycerol | — | — | — | 0.1 | 1 | 5 |
| Dipropylene | 0.1 | 1 | 5 | — | — | — |
| glycol | | | | | | |
| pH | 6.5 | 6.5 | 6.5 | 6.5 | 6.5 | 6.5 |
| Stability (1-4) | 1 | 1 | 3 | 1 | 1 | 1 |
| Log Reduction | — | — | 7.5 | — | — | 7.5 |
| (MBEC) | | | | | | |

Table 13 shows the effect of increasing concentrations of dipropylene glycol (0.1%; v15.6), (1%; v15.7) (5%; v15.8) and glycerol (0.1%; v14.6), (1%; v14.7) and (5%; v14.8).
(Amounts are % w/v; — shows that MBEC was not tested).

### Results

Stable Formulations with sufficient efficacy against biofilm: v11.1.1, v11.1.3, v11.2.1, v11.2.3, v11.3.3, v13.4, v13.4.1, v13.5, v14.1, v7.1, v7.2, v14.8. These formulations were stable for the test period with complete eradication of biofilms when tested using the MBEC assay.

Stable formulations with insufficient efficacy against biofilm: v11.3.1, v11.4.1. Whilst these formulations were stable for the test period they did not show complete eradication of biofilms when tested using the MBEC assay.

Unstable solutions with sufficient efficacy: v12.3, v15.1, v15.8. These formulations showed complete eradication of biofilm however were not stable for the test period.

Unstable solutions with insufficient efficacy: v13.1, v13.2, v13.3. These solutions were neither stable for the test period or showed complete eradication of biofilms when tested using the MBEC assay.

### Example 3

A further formulation of the invention, referred
to herein as v9 can be found in Table 14 below:

| Ingredients | CAS | Quantity (%) |
|---|---|---|
| Alkyl Dimethyl Benzyl Ammonium | 68424-85-1 | 0.1 |
| Chloride (Benzalkonium Chloride) | | |
| N,N-Didecyl-N,N- | 7173-51-5 | 0.1 |
| dimethylammoniumchloride | | |
| (Didecyldimonium chloride) | | |
| Dimethyloctadecyl[3- | 27668-52-6 | 0.1 |
| (trimethoxysilyl)propyl]ammonium | | |
| chloride | | |
| Chlorohexidane Digluconate | 18472-51-0 | 0.1 |
| Cocamidopropyl Betaine | 97862-59-4 | 0.36 |
| Tetrasodium EDTA | 6381-92-6 | 0.05 |
| Other ingredients (carriers for raw | | 0.3 |
| materials) | | |
| Water | | 98.99 |
| pH | | 6.5 |

### Antimicrobial Testing

v9 has been systematically tested in-house. The purpose of the testing has been to evaluate the antimicrobial and antibiofilm efficacy of v9 against Pseudomonas aeruginosa and Staphylococcus aureus, etiological agents for chronic wound infections.

In house testing has revealed that v9 is an effective antimicrobial agent. This was confirmed by antimicrobial susceptibility testing following ISO 20776-1:2019 and antibiofilm efficacy testing following ASTM E2799.

### Susceptibility Testing

Minimal inhibitory concentration (MIC) and Minimum Bactericidal Concentration (MBC) of v9 were determined for both Pseudomonas aeruginosa and for Staphylococcus aureus using a method recommended by the Clinical and Laboratory Standards Institute (CLSI), the European Committee on Antimicrobial Susceptibility Testing (EUCAST; www.Eucast.org) and ISO 20776-1:2019. A MIC of 15.6 ppm and 0.79 ppm for Pseudomonas aeruginosa and for Staphylococcus aureus and an MBC of 45.1 ppm and 1.44 ppm for Pseudomonas aeruginosa and for Staphylococcus aureus were achieved by v9.

TABLE 15
MIC and MBC results of v9 using EUCAST test methods.

| Microorganisms | MIC (ppm) | MBC (ppm) |
|---|---|---|
| Pseudomonas aeruginosa ATCC | 15.6 | 45.1 |
| 15442 | | |
| Staphylococcus aureus ATCC | 0.79 | 1.44 |
| 6538 | | |

### Minimum Biofilm Eradication Concentration (MBEC) (ASTM E2799)

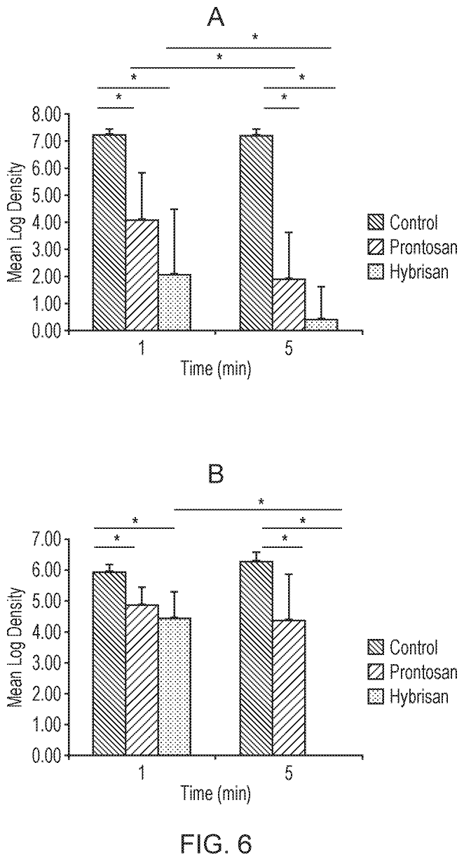

Further studies in-house confirmed that v9 is an effective antibiofilm product using the MBEC ASTM E2799 protocol with wound microorganisms. Pseudomonas aeruginosa ATCC 15442 biofilms when grown for 24 h and treated with v9 produced a 5.2 and a 6.8 log reduction while Prontosan® indicated a 3.2 and 5.3 log reduction at 1 and 5 min respectively (FIG. 6A). These were both significant reductions in biofilm biomass. Staphylococcus aureus ATCC 6538 biofilms when grown for 24 h and treated with v9 produced a 1.5 and a 6.15 log reduction while Prontosan® indicated a 1.1 and 2.3 log reduction at 1 and 5 min respectively (FIG. 6B). Efficacy improved depending on contact time and significant reductions in biofilm biomass were seen for both products. However, v9 consistently outperformed Prontosan®.

### Impact of v9 on Static Biofilms; Established (24 h), and Regrowth

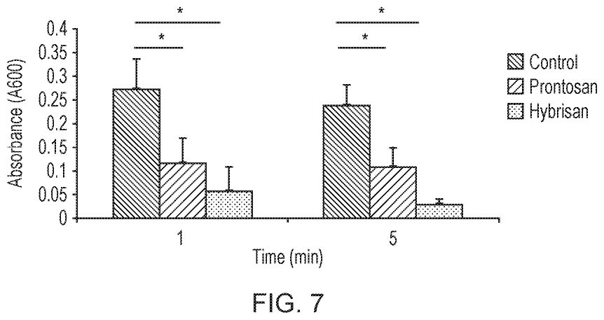

Established biofilms of Pseudomonas aeruginosa ATCC 15442 were grown for 24 h and treated with v9 or Prontosan® for 1 min and 5 min and compared to a no-treatment control. A significant reduction was seen for both v9 and Prontosan®. At both 1 and 5 min a greater reduction was seen for Hybrisan Technology (77 and 88%) than for Prontosan® (54 and 57%) respectively (FIG. 7).

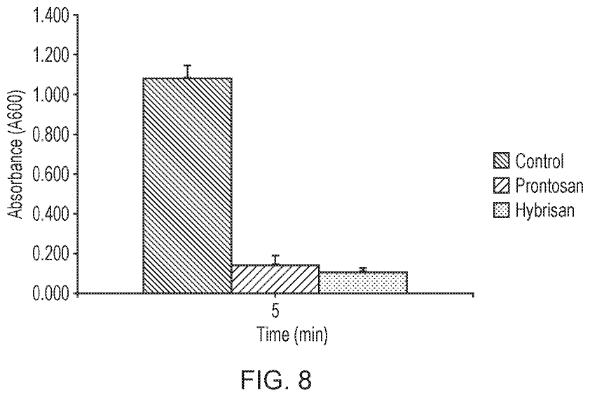

A further study was conducted where biofilms (24 h) were grown and treated with either v9 or Prontosan® for 5 min and then incubated for a further 24 h in the presence of growth media. This has been conducted to evaluate the latent efficacy of either treatment. A significant reduction of growth was seen for both v9 and Prontosan®, although there was less regrowth seen for v9, when compared to the no treatment control. Regrowth following treatment for v9 and Prontosan® was 10% and 13% respectively when compared to the untreated control (FIG. 8).

### REFERENCES

Salisbury A M, Woo K, Sarkar S, Schultz G, Malone M, Mayer D O, Percival S L. Tolerance of Biofilms to Antimicrobials and Significance to Antibiotic Resistance in Wounds. Surg Technol Int. 2018 Nov. 11; 33:59-66. PMID: 30326137

1. An antimicrobial composition comprising:
(i) one or more water soluble organosilanes at a concentration of about 0.01% to about 0.4% w/v;
(ii) one or more quarternary ammonium compounds at a concentration of about 0.01% to 0.5% w/v; and
(iii) one or more non-ionic or amphoteric or sarcosine anionic surfactants at a concentration of about 0.05% to about 1% w/v.

2. The antimicrobial composition according to claim 1, wherein the water soluble organosilane is a quarternary ammonium silane.

3. The antimicrobial composition according to claim 2, wherein the quaternary ammonium silane is one or more of dimethyloctadecyl[3-(trimethoxysilyl)propyl]ammonium chloride and 3-(trihydroxysilyl)propyldimethyloctadecylammoniumchloride.

4. The antimicrobial composition according to claim 1, wherein the quaternary ammonium compound is selected from one or more of benzalkonium chloride (BAC), didecyldimethylammonium chloride (DDAC), benzethonium chloride, tetradonium bromide, cetrimonium bromide, laurtrimonium bromide, cetalkonium chloride and cetrimonium chloride.

5. The antimicrobial composition according to claim 1, wherein the non-ionic or amphoteric or sarcosine anionic surfactant is selected from one or more of Cocomidopropyl betaine, Polyethyleneglycol lauryl ether, Poloxamer 188, Polysorbate 80, PEG-7 Glyceryl Cocoate, PEG-7 oleamide, 2-[4-(2,4,4-trimethylpentan-2-yl)phenoxy]ethanol, Polysorbate 20, Poloxamer 407, Cocomidopropylamine oxide and lauramidopropyl betaine.

6. The antimicrobial composition according to claim 1, wherein the composition additionally comprises a chelating agent.

7. The antimicrobial composition according to claim 6, wherein the chelating agent concentration is from about 0.01% to about 0.2% w/v.

8. The antimicrobial composition according to claim 6, wherein the chelating agent is selected from one or more of disodium EDTA, Trisodium EDTA and tetrasodium EDTA.

9. The antimicrobial composition according to claim 1, wherein the composition additionally comprises a polymeric biguanide.

10. The antimicrobial composition according to claim 9, wherein the polymeric biguanide is selected from one or more of chlorhexidine and polyhexamethylene biguanide.

11. The antimicrobial composition according to claim 1, wherein the composition has a pH of about 4.5 to about 8.5.

12. The antimicrobial composition according to claim 1, wherein the composition further comprises one or more pharmaceutically acceptable carriers and/or excipients.

13. The antimicrobial composition according to claim 1, wherein the composition is formulated for topical administration.

14. The antimicrobial composition according to claim 1, wherein the composition is antibacterial.

15. The antimicrobial composition according to claim 1, wherein the composition is anti-biofilm.

16. A method of making the antimicrobial composition according to claim 1, the method comprising:
(a) combining:
(i) a water soluble organosilane at a concentration of about 0.01% to about 0.4% w/v;
(ii) one or more quarternary ammonium compounds at concentration of about 0.01% to 0.5% w/v;
(iii) a non-ionic or amphoteric surfactant at a concentration of about 0.05% to about 1% w/v; and
(iv) water to form a solution; and
(b) adjusting the pH of the solution to about 4.5 to about 8.5.

17.-22. (canceled)

23. A method of treating and/or preventing a skin and/or wound infection on a patient, the method comprising administering the antimicrobial composition according to claim 1 to the skin and/or to the wound of the patient.

24. The method according to claim 23, wherein the infection is a bacterial infection.

25. The method according to claim 24, wherein the bacterial infection comprises a biofilm.

26. The method according to claim 23, wherein the wound is an open wound.

27. The method according to claim 23, wherein the wound is acute or chronic.
